(19) United States
(12) Patent Application Publication
(10) Pub. No.: US 2018/0210000 A1
(43) Pub. Date: Jul. 26, 2018

(54) LABORATORY

(71) Applicant: Roche Diagnostics Operations, Inc., Indianapolis, IN (US)

(72) Inventor: Hans van Mierlo, Waalwijk (NL)

(73) Assignee: Roche Diagnostics Operations, Inc., Indianapolis, IN (US)

(21) Appl. No.: 15/862,261

(22) Filed: Jan. 4, 2018

(30) Foreign Application Priority Data
Jan. 24, 2017 (EP) 17152913.4

(51) Int. Cl. G01N 35/04; G01N 33/487; B01L 9/06

(52) U.S. Cl. CPC G01N 35/04; G01N 33/4875; G01N 2035/0474; B01L 2200/025; B01L 9/06

(57) ABSTRACT

A laboratory with a laboratory sample distribution system being at least partially arranged in an underfloor chamber is presented.

### CROSS-REFERENCE TO RELATED APPLICATIONS

This application is based on and claims priority to EP 17152913.4, filed Jan. 24, 2017, which is hereby incorporated by reference.

### BACKGROUND

The present disclosure generally relates to a laboratory comprising a laboratory room.

Such a laboratory may typically comprise a number of analytical stations and a laboratory sample distribution system adapted to distribute sample container carriers or sample containers between the stations. Typical laboratory sample distribution systems allow for high throughput and for reliable operation.

It has been found that in typical laboratories problems may arise due to complex laboratory layouts. For example, the laboratory sample distribution system should be able to distribute samples or sample containers between the stations as flexibly as possible, which requires using a substantial part of the laboratory space for a sufficiently large transport plane. However, this requirement deteriorates accessibility of the stations by operators and makes it difficult to implement emergency escape routes.

Therefore, there is a need for a laboratory that is optimized regarding the problems mentioned above.

### SUMMARY

According to the present disclosure, a laboratory is presented. The laboratory can comprise a laboratory room, an underfloor chamber located below the laboratory room, a number of pre-analytical, analytical and/or post-analytical stations, a number of sample container carriers configured to carry sample containers comprising samples to be processed by the stations, and a laboratory sample distribution system configured to distribute the sample container carriers between the stations. The laboratory sample distribution system can comprise a transport plane, a driver configured to move the sample container carriers on the transport plane, and a control device configured to control the movement of the sample container carriers on top of the transport plane by driving the driver such that the sample container carriers move along corresponding transport paths. The laboratory sample distribution system can be at least partially arranged inside the underfloor chamber.

Accordingly, it is a feature of the embodiments of the present disclosure to provide for an optimized laboratory. Other features of the embodiments of the present disclosure will be apparent in light of the description of the disclosure embodied herein.

### BRIEF DESCRIPTION OF THE DRAWING

The following detailed description of specific embodiments of the present disclosure can be best understood when read in conjunction with the following drawing, where like structure is indicated with like reference numerals and in which:

FIG. 1 illustrates a laboratory according to an embodiment of the present disclosure.

### DETAILED DESCRIPTION

In the following detailed description of the embodiments, reference is made to the accompanying drawing that form a part hereof, and in which are shown by way of illustration, and not by way of limitation, specific embodiments in which the disclosure may be practiced. It is to be understood that other embodiments may be utilized and that logical, mechanical and electrical changes may be made without departing from the spirit and scope of the present disclosure.

A laboratory is presented. The laboratory can comprise a laboratory room or laboratory space and an underfloor chamber located below or under the laboratory room or laboratory space. The laboratory can comprise a number (e.g. 1 to 50) of pre-analytical, analytical and/or post-analytical stations.

The laboratory can comprise a number (e.g. 1 to 10.000) of sample container carriers configured to carry the sample containers comprising samples to be processed by the stations.

The laboratory can comprise a laboratory sample distribution system configured to distribute the sample container carriers between the stations. The laboratory sample distribution system can comprise a transport plane, a driver configured to move the sample container carriers on the transport plane, and a control device configured to control the movement of the sample container carriers on top of the transport plane by driving the driver such that the sample container carriers move along corresponding transport paths. The laboratory sample distribution system can be at least partially arranged inside the underfloor chamber.

By use of the laboratory, at least a part of the laboratory sample distribution system can be arranged inside the underfloor chamber, thus leaving free space in the laboratory room that can be used, for example, to provide for emergency exit routes or to allow accessibility of the stations by operators.

Further, distribution of consumables can be simplified as they can, at least partially, be provided using the underfloor chamber.

The underfloor chamber can, in principle, be provided by any kind of hollow interior below the laboratory room. For example, the underfloor chamber can be provided under the whole laboratory room, or under a part of the laboratory room.

The stations may be arranged adjacent to the laboratory sample distribution system.

Pre-analytical stations may be adapted to perform any kind of pre-processing of samples, sample containers and/or sample container carriers.

Analytical stations may be adapted to use a sample or part of the sample and a reagent to generate a measuring signal, the measuring signal indicating if and in which concentration, if any, an analyte exists.

Post-analytical stations may be adapted to perform any kind of post-processing of samples, sample containers and/or sample container carriers.

The pre-analytical, analytical and/or post-analytical stations may comprise at least one of a decapping station, a recapping station, an aliquot station, a centrifugation station, an archiving station, a pipetting station, a sorting station, a tube type identification station, a sample quality determining station, an add-on buffer station, a liquid level detection station, and a sealing/desealing station. The sample container carriers can be regarded as part of the laboratory sample distribution system. However, the sample container carriers can also be regarded as separate from the sample distribution system.

Sample containers can typically be designed as tubes made of glass or transparent plastic and can typically have an opening at an upper end. The sample containers can be used to contain, store and transport samples such as blood samples or chemical samples.

The transport plane can also be denoted as transport surface. The transport plane can support the sample container carriers, what can also be denoted as carrying the sample container carriers.

The control device can typically be a microprocessor, a microcontroller, a field programmable gate array, a standard computer, or a similar device. In a typical embodiment, the control device can comprise a processor and storage. Program code can be stored in the storage in order to control the behavior of the processor when the storage code is executed on the processor.

The sample container carriers can typically be configured to move in two dimensions on the transport plane.

According to an embodiment, the transport plane can be arranged inside the underfloor chamber. The transport plane can be arranged completely inside the underfloor chamber. This can allow for solely using the underfloor chamber for transporting the sample container carriers.

According to an embodiment, the transport plane can comprise a number (e.g. 1 to 100) of sections arranged above or outside the underfloor chamber. Each section of the number of sections arranged above or outside the underfloor chamber may be assigned to at least one of the number of pre-analytical, analytical and/or post-analytical stations. The transport plane may comprise a number (e.g. 1 to 100) of sections arranged inside the underfloor chamber. This can allow for a suitable distribution of transport plane sections between the laboratory room and the underfloor chamber.

According to an embodiment, the laboratory sample distribution system can further comprise a vertical transporter. The vertical transporter can be configured to transport sample container carriers or sample containers between the transport plane and a location having a vertical level different from a vertical level of the transport plane. Such a vertical transporter can allow for a distribution of a sample container carrier from a transport plane to a station even if the station is located at another level than the transport plane. For example, the station can be placed inside the laboratory room and the transport plane or the relevant section of the transport plane can be placed inside the underfloor chamber, or vice versa.

The vertical transporter can also be used to transport sample containers or sample container carriers between sections of the transport plane having different vertical levels.

According to an embodiment, the underfloor chamber can be covered by a number (e.g. 1 to 1000) of releasable floor tiles. This can allow for easy access to the underfloor chamber by simply removing the floor tiles or a part of the floor tiles.

According to an embodiment, the floor tiles can be embodied to provide a walk-in ground floor. This can allow for easy access of operators to locations in the laboratory room. The tiles covering the underfloor chamber can be used as a walking floor. Also a higher load resistance can be possible, if needed.

According to an embodiment, some or all of the floor tiles can be optically transparent. This can allow for viewing operation of the parts of the laboratory sample distribution system arranged below the laboratory room, which would otherwise not be viewable during normal operation. For example, errors, if any, can be detected.

According to an embodiment, the underfloor chamber can comprise a number (e.g. 1 to 100) of supplies such as, for example, electrical or fluid supplies. The supplies may supply at least one of the number of pre-analytical, analytical and/or post-analytical stations, in particular with electrical energy or with a fluid, in particular with a coolant or reagent. This can allow for at least partially or wholly providing supplies using the underfloor chamber, which can also save limited space in the laboratory room.

According to an embodiment, the laboratory can comprise a plurality of underfloor chambers. The laboratory sample distribution system can be at least partially arranged inside the underfloor chambers. This can allow distribution of the laboratory sample distribution system over a plurality of underfloor chambers.

According to an embodiment, the laboratory can comprise sealant configured to seal the underfloor chamber liquid-tight. This can prevent liquids flowing into the underfloor chamber; those liquids possibly can prevent safe operation or contaminate samples to be analyzed. For example, such sealant can be arranged around the floor tiles.

According to an embodiment, the laboratory sample distribution system can comprise underfloor storage to temporarily store a number (e.g. 1 to 10.000) of sample container carriers, e.g. empty sample container carriers, or sample containers. The underfloor storage can be arranged inside the underfloor chamber. The underfloor storage may comprise a sample container carrier storing device, e.g. arranged adjacent to the transport plane, or embodied as a sector on the transport plane. This can allow for at least temporarily storing sample container carriers using the underfloor chamber, which can also save space in the laboratory room that would otherwise be needed for storage in the laboratory room.

According to an embodiment, the driver can be embodied as electro-magnetic actuators and each sample container carrier can comprise a magnetically active device, for example a permanent magnet. This can allow for efficient and reliable operation.

According to a further embodiment, the driver can be formed as wheels driven by electric motors located in the sample container carriers and controllable by the control device.

According to an embodiment, the transport plane can be arranged solely inside the underfloor chamber, at least one station of the stations can be arranged in the underfloor chamber, and at least one station of the stations can be arranged above the underfloor chamber. This can allow for a suitable distribution of the stations between the underfloor chamber and the laboratory room.

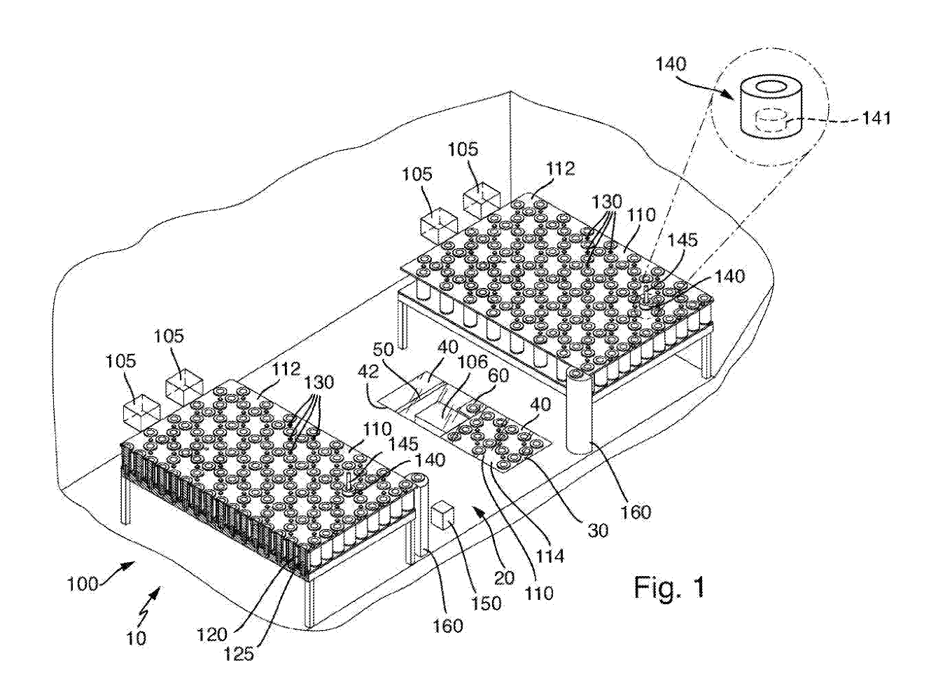

Referring initially to FIG. 1, FIG. 1 shows a laboratory 10. The laboratory 10 can comprise a laboratory room 20 and an underfloor chamber 30 arranged below or under the laboratory room 20. The underfloor chamber 30 can be accessible by two removable floor tiles 40.

The floor tiles 40 can be optically transparent so that it can be possible to see into the underfloor chamber 30. The floor tiles 40 can be dimensioned to withstand the weight of a person standing on it, so that the floor tiles 40 can provide for a walk-in floor.

The floor tiles 40 can be surrounded by a sealant 42 being liquid-tight so as to prevent liquids or other contaminants from entering the underfloor chamber 30.

The laboratory 10 can comprise a laboratory sample distribution system 100 and a number of pre-analytical, analytical and/or post-analytical stations 105, 106 arranged adjacent to the laboratory sample distribution system 100. Self-evidently, more than the five stations 105, 106 schematically depicted in FIG. 1 may be comprised in the laboratory sample distribution system 100. In the shown example, four stations 105 can be arranged in the laboratory room 20 and one station 106 can be arranged in the underfloor chamber 30.

The laboratory sample distribution system 100 can comprise a transport plane 110 below in which a plurality of electro-magnetic actuators in the form of electromagnets 120 can be positioned. The electromagnets 120 can be implemented as solenoids having solid ferromagnetic cores 125.

The transport plane 110 can have sections 112 provided in the laboratory room 20 and sections 114 provided in the underfloor chamber 30. Thus, the transport plane 110 can be distributed between the laboratory room 20 and the underfloor chamber 30.

Sample container carriers 140 respectively comprising a magnetically active device 141 in form of a permanent magnet can be positioned on the transport plane 110 and can be moved by the electromagnets 120. While it can be understood that a plurality of sample container carriers 140 can be positioned on the transport plane 110, due to simplicity only one respective sample container carrier 140 on each of the two sections 112 of the transport plane 110 in the laboratory room 20 is depicted in FIG. 1. Each sample container carrier 140 can hold a respective sample container 145, in which a sample to be analyzed can be contained.

The laboratory sample distribution system 100 can be configured to transport the sample container carriers 140 and/or the sample containers 145 between the laboratory stations 105, 106. The laboratory stations 105, 106 can be positioned adjacent to the transport plane 110 such that a sample container carrier 140 can be used to transport a sample contained in the sample container 145 to a respective laboratory station 105, 106.

A plurality of Hall-sensors 130 can be arranged such that positions of respective sample container carriers 140 on the transport plane 110 can be detected.

The laboratory sample distribution system 100 can further comprise a control device 150. The control device 150 can be configured to control movement of the sample container carriers 140 on the transport plane 110 by driving the electromagnets 120 such that the sample container carriers 140 independently and simultaneously move along corresponding transport paths.

The laboratory sample distribution system 100 can further comprise two vertical transporters 160. As shown, one respective vertical transporter 160 can be arranged besides each of the two sections 112 of the transport plane 110 in the laboratory room 20.

The vertical transporters 160 can be embodied as vertically moving planes on which a sample container carrier 140 can be positioned and transported to a different level. In the present case, each vertical transporter 160 can be configured to transport a sample container carrier 140 between a section 112 in the laboratory room and the section 114 in the underfloor chamber 30. Thus, sample container carriers 140 and sample containers 145 can be moved on the entire transport plane 110. For example, a sample container carrier 140 carrying a sample container 145 can be moved from one of the two sections 112 in the laboratory room 20 to the section 114 in the underfloor chamber 30 and can then be moved further to the other section 112 in the laboratory room 20.

For holding the sample container carrier 140 to be transported, each vertical transporter 160 can comprise an electro-magnetic actuator similarly to the electro-magnetic actuators 120.

Given the functionality just described, sample container carriers 140 can be distributed freely over the entire transport plane 110 using sections 112, 114 both in the laboratory room 20 and in the underfloor chamber 30. However, in contrast to systems known in the prior art, an empty space can be left in the laboratory room 20 between the two sections 112 of the transport plane 110. This can, for example, be used for maintenance purposes or as an emergency escape route.

The shown station 106 positioned in the underfloor chamber 30 can also be used similarly to the stations 105 positioned in the laboratory room 20.

An underfloor storage 60 can be arranged inside the underfloor chamber 30 to temporarily store sample container carriers 140. The underfloor storage 60 can be embodied as a horizontal extension of the section 114.

In addition, a supply 50 in form of a fluid pipe can be arranged inside the underfloor chamber 30. The fluid pipe can be used to supply fluids to the stations 105, 106. Usage of the underfloor chamber 30 for this purpose can have the advantage that these supply 50 does not occupy space in the laboratory room 20. Similarly, the underfloor chamber 30 can be used to provide other consumables, electricity or data communications means, etc.

It is noted that terms like “preferably,” “commonly,” and “typically” are not utilized herein to limit the scope of the claimed embodiments or to imply that certain features are critical, essential, or even important to the structure or function of the claimed embodiments. Rather, these terms are merely intended to highlight alternative or additional features that may or may not be utilized in a particular embodiment of the present disclosure.

Having described the present disclosure in detail and by reference to specific embodiments thereof, it will be apparent that modifications and variations are possible without departing from the scope of the disclosure defined in the appended claims. More specifically, although some aspects of the present disclosure are identified herein as preferred or particularly advantageous, it is contemplated that the present disclosure is not necessarily limited to these preferred aspects of the disclosure.

I claim:

1. A laboratory, the laboratory comprising:
a laboratory room;
an underfloor chamber located below the laboratory room;
a number of pre-analytical, analytical and/or post-analytical stations;
a number of sample container carriers configured to carry sample containers comprising samples to be processed by the stations; and
a laboratory sample distribution system configured to distribute the sample container carriers between the stations, wherein the laboratory sample distribution system comprises,
a transport plane;
a driver configured to move the sample container carriers on the transport plane, and
a control device configured to control the movement of the sample container carriers on top of the transport plane by driving the driver such that the sample container carriers move along corresponding transport paths, wherein the laboratory sample distribution system is at least partially arranged inside the underfloor chamber.

2. The laboratory according to claim 1, wherein the transport plane is arranged inside the underfloor chamber.

3. The laboratory according to claim 1, the transport plane comprises a number of sections arranged above the underfloor chamber.

4. The laboratory according to claim 1, wherein the laboratory sample distribution system further comprises a vertical transporter, wherein the vertical transporter is configured to transport sample container carriers or sample containers between the transport plane and a location having a vertical level different from a vertical level of the transport plane.

5. The laboratory according to claim 1, wherein the underfloor chamber is covered by a number of releasable floor tiles.

6. The laboratory according to claim 5, wherein the floor tiles are embodied to provide a walk-in ground floor.

7. The laboratory according to claim 5, wherein some or all of the floor tiles are optically transparent.

8. The laboratory according to claim 1, wherein the underfloor chamber comprises a number of supplies.

9. The laboratory according to claim 1, wherein the number of supplies are electrical or fluid supplies.

10. The laboratory according to claim 1, further comprises
a plurality of underfloor chambers, wherein the laboratory sample distribution system is at least partially arranged inside the underfloor chambers.

11. The laboratory according claim 1, further comprises
sealant configured to seal the underfloor chamber liquid-tight.

12. The laboratory according to claim 1, wherein the laboratory sample distribution system comprises underfloor storage to temporarily store a number of sample container carriers or sample containers, wherein the underfloor storage is arranged inside the underfloor chamber.

13. The laboratory according to claim 1, wherein the driver is embodied as electro-magnetic actuator and each sample container carrier comprises a magnetically active device.

14. The laboratory according to claim 1, wherein the transport plane is arranged solely inside the underfloor chamber, at least one of the stations is arranged in the underfloor chamber, and at least one of the stations is arranged above the underfloor chamber.
